(19) United States
(12) Patent Application Publication
(10) Pub. No.: US 2004/0267178 A1
(43) Pub. Date: Dec. 30, 2004

(54) Cervical collar

(76) Inventor: Petra Benckendorff, Hamburg (DE)

Correspondence Address: Friedrich Kueffner, Suite 910, 317 Madison Avenue, New York, NY, 10017, US

(21) Appl. No.: 10/869,314

(22) Filed: Jun. 16, 2004

(30) Foreign Application Priority Data
Jun. 24, 2003 (DE) 203 09 801.3

(51) Int. Cl. A61F 5/00

(52) U.S. Cl. 602/18

(57) ABSTRACT

In order to make possible a tension free wearing, in particular in the larynx area, of a cervical collar made of a strip-shaped blank (20) with a removable closure, the blank (20) of the cervical collar (10) has at the ends an adhesion or Velcro closure (30, 31) as closing parts, whereby each end (20a, 20b) of the blank (20) is provided with at least one slit or notch (22, 23) in blank longitudinal direction by configuring at least two tongue-like end sections (21, 21a; 22, 22a), whereby the tongue-like end sections (21, 21a; 22, 22a) of the blank (20) bear the closing parts (30, 31).

### TECHNICAL FIELD

This invention relates to a cervical collar with cervical supporting properties made of a strip-shaped blank with a detachable closure.

### BACKGROUND OF THE INVENTION

Cervical supports and so-called cervical collars are known. Fabricated ortheses for load relieving the cervical spine and supporting the head are understood herewith. Cervical supports are used in particular for a so-called whiplash injury of the cervical spine by a traffic accident. During a rear-end collision, the head is flung forward and backward due to the acting forces, whereby the physiologic moving scale of the cervical spine is exceeded. This being, there appear distorsions or tearings of the soft tissue structures and osseous impressions which lead to a restriction of the cervical spine mobility. As a therapy for the immobilization, a cervical support is often used which principally constitutes a solid ring-shaped cervical collar with chin bearing. This cervical collar is designed in such a way that it relieves the neck muscles and supports the head. The disadvantage of the cervical support consists in the fact that, when wearing it a longer time, it can come to a weakening of the shoulder neck muscles which finally results in the fact that the patients cannot hold the head autonomously any longer.

Thus, instead of the cervical support known by the prior art, the so-called Schanz's dressing is recommended in many cases. This dressing is made of a cooton roll, the width of which corresponds to the neck height, which is simultaneously wrapped around the neck with wide ideal or cotton cambric bandages in such a manner that the rollers continuously fix and compress the unrolling cotton. The dressing bears widely on the pulled down shoulders and the breastbone and support the head on all sides in the height. Eventually, the cervical collar is reinforced by a strong bandage which is wrapped over.

Since the temporary removal of the cervical collar is wished for avoiding the weakening of the muscles, ready-made cervical collar have been proposed which are much lighter and which can be applied by the patient himself. These cervical collars are made of a rigid non-woven ply with a reinforcement envelope as a foam envelope. The disadvantage of these cervical collars consists in that their fitting is far from being as good and firm as wished.

The U.S. Pat. No. 4,819,622 knows a cervical collar made of several layers folded on each other to a strip-shaped blank and sewn together on their edges made of a textile material, for example of terry cloth, the ends of which are provided with a detachable closure, in particular with a Velcro closing. Since the layers of the cervical collar which are folded on each other are sewn together only on the edges, this cervical collar does no have a sufficient inherent rigidity in order to achieve a sufficient supporting effect since there is no sufficient rigidity of the layers in the middle sections of the folded layers. Add to this that this cervical collar is made of a textile material which has extendible properties because of its woven textile and binding structure.

Furthermore, the DE-A-44 95 250 knows a cervical collar made of a strip-shaped blank with a detachable closure which is made of several molleton layers which are close meshedly sewn together. Thus, a day and night wearable cervical collar or cervical support with a high wearing comfort is supposed to be created which can be used not only for the support by injuries and for the cervical spine syndrom but also by travellers, for example flight passengers, on long distances for improving the posture of the head, in particular during the resting breaks and the position during the sleep in seating position or in other positions.

The known cervical collars are provided at the ends with parts of an adhesive closure or Velcro closing and have relatively big widths so that they are supported, when applied, on the chin side area and in the lateral neck area which results in the fact that these cervical collars can be worn on both sides, which is attributed in particular to the anatomic configuration. Add to this that the closure area situated in the larynx area is often felt as being annoying, in particular when men with big Adam's apples or persons which have larynx swellings or goiter like enlargements because of thyroid diseases wear such cervical collars, which often results in constrictions or is felt as a constriction.

### SUMMARY OF THE INVENTION

Thus, the aim of the invention is to create a cervical collar of the above mentioned type which makes possible a tension free wearing, in particular in the larynx area and with which the above mentioned disadvantages are avoided so that the cervical collar wearer does not feel any constriction in the larynx area and any oppression feeling when the cervical collar is applied. Furthermore, the cervical collar should be wearable on both sides.

This aim is achieved with a cervical collar with the characteristics indicated in claim 1.

According to the invention, the cervical collar is configured accordingly so that its blank has at the ends an adhesion or Velcro closure as closing parts, whereby each end of the blank is provided with at least one slit or notch in blank longitudinal direction by configuring at least two tongue-like end sections, whereby the tongue-like end sections of the blank bear the closing parts.

Because of this configuration, a cervical collar with a high wearing comfort is created which consists in particular in that the closing parts of the applied cervical collar situated in the larynx area press neither onto the larynx nor on any larynx thickening. The feeling of a constriction is avoided; the wearing of the cervical collar does not constitute any strain any longer; there do not appear any pressure points and since the closing parts are situated in the larynx area, the application and the removal of the cervical collar is easy and can also be done by older people.

Because of the special blank shape of the cervical collar in which the blank is configured relatively narrow, whereby the width of the blank can preferably correspond approximately to a sixth of the length of the blank, and has, in the area of its both longitudinal sides, two arc-shaped elevations situated in the lateral neck area, when the cervical collar is applied, the cervical collar can be worn on both sides, which is particularly advantageous if at least one side of the blank of the cervical collar is provided with a hyperemiazing or heat retaining lining.

Improvements of the invention are described in the subclaims.

### BRIEF DESCRIPTION OF THE DRAWINGS

Embodiments of the invention are represented in the drawings.

FIG. 1 shows a view of the cervical collar.

FIG. 2 shows the outer side of the cervical collar.

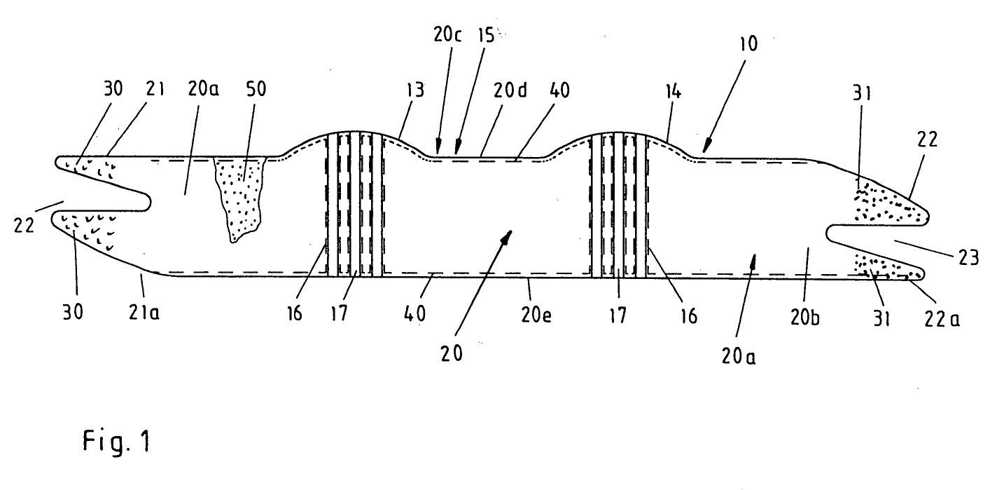

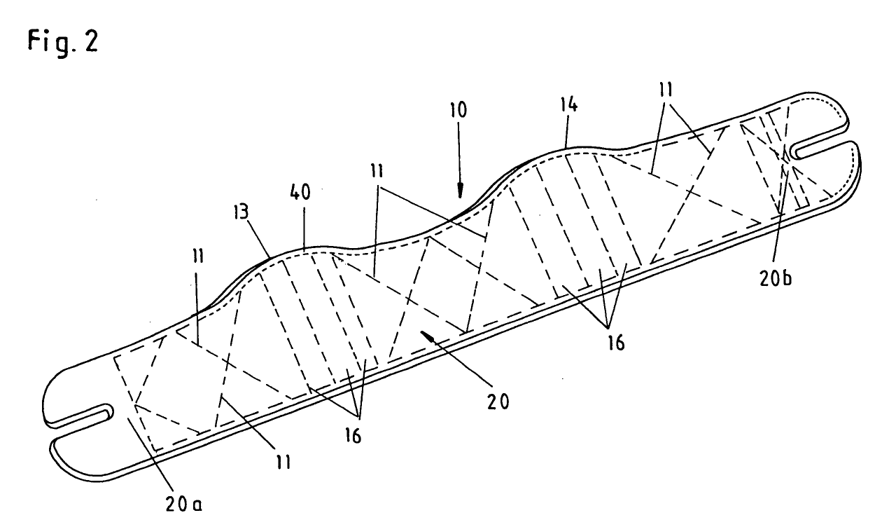

The cervical collar 10 represented in FIG. 1 and 2 consists for example of several layers of molleton which are close meshedly sewn together with lozenged seams 11. These seams or tucks increase the inherent stability of the base material molleton. For the molleton, the matter is of a soft plain weaved cotton fabric brushed on both sides. However, differently configured materials can also be used for the production of the cervical collar.

The cervical collar 10 consists of a relatively narrow blank 20. Preferably the width of the blank 20 is a sixth of the total length of the blank.

Velcro closing elements 30 and 31 which make possible an easy opening and closing of the cervical collar are provided on the end sides. The cervical collar has a width b which corresponds approximately to the length of the neck part to be supported. The length of the cervical collar which is represented in the unrolled state corresponds approximately to the neck circumference. The cervical collar has two elevations 13 and 14 placed the one besides the other which bear on the neck parts or on the lateral neck area and support here, when the cervical collar is applied while the area 15 bears in the neck. Pockets 16 with smooth inner walls which extend over the whole width are provided in these raised areas, pockets in which stiffening little rods 17 are stuck. No material tucks are provided in the area of the pockets 16. A pocket 16 with a stiffening little rod 17 can be provided in each area, or several pockets 16, for example three pockets 16, with stiffening little rods 17 can also be provided. In this case, respectively three pockets with three stiffening little rods are situated in the cervical collar. On the outside, layers are sewn together over the pockets and the stiffening little rods so that optimal stiffenings are created just in the areas 13 and 14. The molleton material is dyed-in. The most various colours can be used here.

The blank 20 of the cervical collar 10 has, in the area of its ends 20a, 20b, the parts 30, 31 of an adhesion or Velcro closing as closure, whereby differently configured closings can also be used.

Each end 20a, 20b of the blank 20 is provided with at least one slit or notch 22, 23 in blank longitudinal direction by configuring at least two tongue-like end sections 21, 21a and 22, 22a.

These tongue-like end sections 22, 22a carry the closing parts 30, 31.

The blank 20 of the cervical collar 10 is provided in its edge area 20c with a padding 40 made of a soft padding material so that, when the cervical collar 10 is applied, there cannot appear any pressure points in the neck area.

Both elevations 13, 14 are configured on one of the two longitudinal sides 20d, 20e of the blank 20; they constitute arc-shaped sections of low height in the lateral neck area. The padding 40 configured in the edge area 20c of the blank 20 also extends over the edge area of both elevations 13, 14.

The length of each elevation 13, 14 corresponds approximately to a tenth of the total length of the blank 20.

At least one side of the blank 20 of the cervical collar 10 is provided with a hyperemiazing or heat retaining lining 50. Since the cervical collar 10 is wearable on both sides because of its configuration, it is possible for the lateral surfaces of the blank 10 to have different colours or fabric types.

The stiffening rods 17 can also be configured as rod-shaped pellets which are made of a plastic adjusted to the respectively wished hardness, for example of silicone rubber or of another appropriate plastic so that, when the cervical collar is applied, a massaging effect is achieved by means of such stiffening rods.

1. Cervical collar made of a strip-shaped blank with a detachable closure, wherein the blank (20) of the cervical collar (10) has at the ends an adhesion or Velcro closure (30, 31) as closing parts and that each end (20a, 20b) of the blank (20) is provided with at least one slit or notch (22; 23) in blank longitudinal direction by configuring at least two tongue-like end sections (21, 21a; 22, 22a), whereby the tongue-like end sections (21, 21a; 22, 22a) of the blank (20) bear the closing parts (30, 31).

2. Cervical collar according to claim 1, wherein the blank (20) of the cervical collar (10) is provided in its edge area (20c) with a padding (40) made of a soft padding material.

3. Cervical collar according to claim 1, wherein the blank (20) is configured narrow and has two arc-shaped elevations (13, 14) of low height in the area of one of its two longitudinal sides (20d, 20e) bearing in the lateral neck area, when the cervical collar (10) is applied.

4. Cervical collar according to claim 1, wherein the length of each arc-shaped elevation (13, 14) approximately corresponds to a tenth of the total length of the blank (20).

5. Cervical collar according to claim 1, wherein at least one side of the blank (20) of the cervical collar (10) is provided with hyperemiazing or heat retaining lining (50).
